(19) United States
(12) Patent Application Publication
(10) Pub. No.: US 2025/0200753 A1
(43) Pub. Date: Jun. 19, 2025

(54) Tracking the Movement of an Object Region in an Examination Tunnel of a Magnetic Resonance Tomography System

(71) Applicant: Siemens Healthineers AG, Forchheim (DE)

(72) Inventors: Julian Wohlers, Erlangen (DE); Johann Sukkau, Herzogenaurach (DE)

(73) Assignee: Siemens Healthineers AG, Forchheim (DE)

(21) Appl. No.: 18/978,078

(22) Filed: Dec. 12, 2024

(30) Foreign Application Priority Data
Dec. 13, 2023 (EP) 23216088.7

(51) Int. Cl. G06T 7/11; A61B 34/20

(52) U.S. Cl. CPC G06T 7/11; A61B 2034/2065; G06T 2207/10088

(57) ABSTRACT

A method for tracking the movement of an object region in an examination tunnel of a magnetic resonance tomography system. The method includes arranging a carrier unit around the object region, such that the position of the carrier unit is linked to the position of the object region, wherein at least one motion sensor is arranged on the carrier unit; defining a starting position of the object region; monitoring the movement acquired by the at least one motion sensor in order to obtain movement data; and determining a current position of the object region relative to the starting position based on the movement data acquired with the at least one motion sensor.

### TECHNICAL FIELD

The disclosure relates to a method for tracking the movement of an object region in an examination tunnel of a magnetic resonance tomography system, a measuring device, and a magnetic resonance tomography system.

### BACKGROUND

Independent of the grammatical term usage, individuals with male, female, or other gender identities are included within the term.

Magnetic resonance tomography is typically based on the capture of tomographic images. One problem is the orientation of the respective tomographic slice relative to an object region to be examined. The prior art has, for some years, included methods for automatically selecting elements in the field of view (FOV), which are based on magnetic resonance measurements and, for example, fixed points of reference (“landmarks”). This method is conventionally known as “automatic alignment” or “auto align.” However, errors repeatedly arise when using automatic selection, resulting, for example, from unusual anatomies or movement of the region to be examined. Movement and rotation of the object region may arise very frequently, particularly when imaging knees or elbows. Movement of the object region between individual scanning steps may be problematic because typical orientation methods are based on a first localization image, which, in the event of movement of the object region, increasingly poorly characterizes the current position as scanning proceeds. Accordingly, the risk of an incorrectly set FOV typically increases in later scanning steps or toward the end of the scan.

The problem is sometimes dealt with by a radiologist manually correcting the position and orientation of the respective slices. However, this solution is time-consuming and often laborious for the radiologist.

For measurements in the field of magnetic resonance tomography, it is advantageous for receive coils to be as close as possible to the location to be measured so as to achieve a good signal-to-noise ratio (SNR). Local coils are, therefore, often placed directly on the object region to be examined. However, it is not simple to precisely determine the position of the local coil. One approach to be found in the prior art (see DE 10 2016 203 255 A1 or US 2017/0248665 A1) involves determining a position of a local coil with the assistance of a magnetic field sensor, wherein knowledge about the magnetic field distribution around an examination tunnel of the magnetic resonance tomography system serves as the basis. However, the magnetic field in the examination tunnel itself is generally uniform, meaning that this position determination cannot be applied directly during a measurement.

Publication EP 4 170 375 A1 relates to determining the position of a coil in the examination tunnel of a magnetic resonance tomography system. In this case, the position of the coil can be determined with the assistance of a marker element and a magnetic resonance tomography measurement. The marker element is disabled for the actual measurement of an object. This makes position determination in an examination tunnel possible. However, disabling the marker element during the actual measurement means that position tracking typically likewise cannot proceed during the actual measurement.

### SUMMARY

It is, accordingly, an object of the present disclosure to provide a possible way of solving at least some of the above-stated problems. In particular, the intention is to find a possible way of tracking the movement of an object region in an examination tunnel of a magnetic resonance tomography system, preferably also during a measurement.

This object is achieved by a method, a measurement device, and a magnetic resonance tomography system. Further features and advantages are revealed by the dependent claims, the description, and the appended figures.

According to a first aspect of the disclosure, a method is provided for tracking the movement of an object region in an examination tunnel of a magnetic resonance tomography system. The method comprises the following steps:
(a) arranging a carrier unit around the object region, such that the position of the carrier unit is linked to the position of the object region, wherein the carrier unit is, in particular, a magnetic resonance tomography body coil, wherein at least one motion sensor is arranged on the carrier unit;
(b) defining a starting position of the object region;
(c) monitoring the movement acquired by the at least one motion sensor in order to obtain movement data; and
(d) determining a current position of the object region relative to the starting position on the basis of the movement data acquired with the at least one motion sensor.

The term object region should be interpreted broadly for the purposes of the disclosure. In general, the object region may be any region of an object. The object region may, for example, be a region of interest (“ROI” for short) or correspond to a region of interest. The object region may be a subregion of the object or the entire region of the object. The object region may, in particular, be a region that is to be examined in the context of a magnetic resonance tomography (MRI) examination. The object may, for example, be part of a living organism, in particular, of an animal or a human. By way of example, the object may be part of a patient. The object may be an anatomical structure and/or an organ. The object may, for example, be a joint, such as a knee or elbow. In the prior art, it is precisely a joint, such as a knee or an elbow, which may lead to difficulties in terms of automatic selection of said region due to the degrees of freedom of movement. It may advantageously be possible, with the method according to the disclosure for tracking movement of the object region, to enable the object region even during a measurement in the examination tunnel of an MRI system, which in itself offers only limited access for movement tracking.

In the context of the method, a carrier unit is arranged around the object region. The carrier unit may be arranged around some or all of the object region. The carrier unit may be, at least in places, of flexible, for example, bendable, configuration. The carrier unit may, for example, be wrapped or folded around the object region. The carrier unit may comprise members joined movably to one another. The carrier unit may be flexible, in particular, windable or foldable, in a preferential direction. Provision may be made for the carrier unit to be flexible in just one preferential direction. With flexibility only in the preferential direction, it is possible, for example, to prevent incorrect arrangement of the carrier unit. The preferential direction relates, in particular, to a direction of rotation or winding direction. In particular, the carrier unit may be rigid in at least one direction that is not the preferential direction. The carrier unit is arranged around the object region in such a way that the position of the carrier unit is linked to the position of the object region. The carrier unit may, for example, be directly or indirectly connected to the object region. An indirect connection may be present, for example, if the carrier unit is connected to an element which is in turn connected to the object region. The carrier unit may, for example, be connected or positionally linked to the object region by being wrapped around it. Advantageously, linking the position of the carrier unit to the position of the object region may enable the position of the object region to be determined by way of the position of the carrier unit. The carrier unit is preferably linked to the object region in such a way that it is translationally invariant and/or rotationally invariant in relation to the object region. The carrier unit may preferably be arranged in accordance with a fixed relative spatial relationship to the object region. For example, a defined location of the carrier unit may be arranged at a defined point relative to the object region. The carrier unit may comprise a marker that can be used to define the position of the carrier unit relative to the object region. The marker may, for example, be or comprise a pattern. For example, the marker may comprise a cross. The marker may, for example, be arranged directly over the object region, in particular, over a fixed point of the object region. The fixed point may be the center of the object region, for example. The pattern may, for example, be arranged over a center of an elbow or a knee. The carrier unit, in particular, the marker, is preferably arranged as close as possible to the object region. An orientation of the carrier unit may be aligned relative to the orientation of the object region. For example, the marker, in particular, a pattern of the marker, may be aligned with the object region.

The carrier unit may, in particular, be a magnetic resonance tomography body coil. A body coil, which is in any case intended to be placed close to the object region, may thus also advantageously be used as a carrier unit. The body coil may be configured to be flexible in at least one direction, such that it can be wrapped around the object region. Alternatively, a carrier unit may also be used, which is not a body coil. For example, not only the body coil but also the carrier unit may be arranged around the object region. The carrier unit that is not a body coil may, for example, be used whether or not a body coil is provided around the object region.

At least one motion sensor is arranged on the carrier unit. The at least one motion sensor may, in particular, be fixed stationarily on the carrier unit. Movement of the motion sensor may thus advantageously be directly linked with movement of the carrier unit and, in particular, with movement of the object region. A motion sensor should be interpreted broadly in the context of the disclosure and is generally a sensor that in some way acquires a movement or a variable linked with a movement. The motion sensor may, for example, be configured to convert movement into an electrical variable. The electrical variable may be a current, a voltage, or a capacitance, for example. The motion sensor may, for example, be configured to acquire a change in location, a speed, and/or an acceleration. The motion sensor may, for example, be an acceleration sensor. The motion sensor, in particular, the acceleration sensor, may be configured to acquire a movement or acceleration in a specific direction and/or a movement or acceleration with direction information. A plurality of motion sensors, in particular, acceleration sensors, may be provided. The sensors may be provided at different locations of the carrier unit. The at least one motion sensor, in particular, acceleration sensor, may be a three-dimensional motion sensor (3D motion sensor) or three-dimensional acceleration sensor (3D acceleration sensor). A three-dimensional sensor should be understood, in the context of the present disclosure, as having a sensor system appropriate for three spatial directions. A 3D motion sensor may separately capture movement data for three spatial directions. Movement in the x direction, movement in the y direction, and movement in the z direction may, in each case, be separately acquired. A 3D acceleration sensor may be configured to detect acceleration separately in each of three individual spatial directions. Movement or acceleration may thus advantageously be acquired together with a direction of the movement or acceleration. A 3D acceleration sensor may, for example, be used to determine an orientation in the earth's gravitational field. The acceleration sensor may thus also be used as an orientation sensor. In addition to the at least one motion sensor, one or more further sensors or at least one further type of sensor may be provided on the carrier unit. The at least one further type of sensor may, in particular, be at least one sensor, which is not a motion sensor. For example, at least one magnetic field sensor or Hall sensor may be provided on the carrier unit. Further sensors may, for example, be used to define the starting position.

A starting position of the object region is preferably defined prior to tracking of the movement with the at least one motion sensor. The starting position may optionally include a starting orientation. In some cases, there may also be the option to define the starting position subsequently. For example, an end position may be determined, and the starting position may be extrapolated back using the acquired movement. The starting position may be defined or determined by a sensor, for example, a position sensor, on the carrier unit. Provision may be made for the starting position of the object region to be determined outside the examination tunnel. It is typically easier to determine a position outside the examination tunnel because not only is visual access, for example, for a camera, simpler there, but the magnetic field is less strong, and there is also more space. The starting position may preferably be automatically defined. Automatic definition may, for example, be defined by way of a detection device. The detection device may comprise a detection unit and an evaluation unit. The evaluation unit may, for example, be part of a control unit of the MRI system or be integrated into the control unit. The detection unit may, for example, be a sensor and/or a camera. The camera may, for example, be an optical camera and/or an infrared camera. The sensor may, for example, be a magnetic field sensor. The magnetic field sensor may estimate a position, in particular, using the magnetic field strength outside the examination tunnel. For example, the starting position may be determined when a patient couch is in a starting position (for example, in a home position).

Movement data is obtained by monitoring the movement acquired by the at least one motion sensor. Movement monitoring should be interpreted broadly. Monitoring may encompass recording and/or an evaluation of the acquired movement. The movement data may be monitored directly after the starting position has been defined. A position of the object region can thus be tracked. The movement data may, in particular, also be collected while the object region is in the examination tunnel. The movement data is preferably monitored without any interruption once the starting position has been determined, at least until an end time. The end time may be the end of a measurement, for example.

The monitored movement data may be used to determine a current position of the object region relative to the starting position. The current position may optionally also encompass a current orientation. Using the movement data, a current position may advantageously be determined in connection with the starting position. The movement data may, for example, be added to the starting position as a relative shift in order to determine a current position of the object region. Advantageously, the use of the motion sensors may thus also enable tracking of a movement in the examination tunnel, which is otherwise difficult to access for these purposes. Movement of a patient or part of the patient, in particular, the object region, may thus be acquired and, if necessary, taken into account. The movement data or the established current position may be used, for example, to carry out current or retroactive movement correction of a scan. For example, a field of view and/or an automatic selection may be adjusted on the basis of the movement data or the established current position. Adjustment can thus advantageously be undertaken directly, and movements can be acquired relatively reliably, including in the examination tunnel. Even spontaneous movements, for example, of a knee or elbow under examination, can, in this way, be detected and automatically taken into account.

The field of view may, in particular, be automatically adjusted, and, generally speaking, it is no longer or, as a rule, no longer necessary for a user to effect manual adjustment.

According to one aspect, at least three motion sensors are fastened to the carrier unit. The at least three motion sensors may, in particular, be at least three acceleration sensors. Having at least three motion sensors enables particularly precise, reliable movement tracking. In particular, inclination or bending of the object region may also be determined.

According to one aspect, the at least one motion sensor is at least one acceleration sensor, wherein the movement data is acceleration data. The current position of the object region is determined by integrating the acceleration data twice over time. An acceleration sensor may be a particularly reliable option for determining movement data also within the examination tunnel. By integrating the acquired acceleration twice, a relative location, i.e., a displacement, of the at least one acceleration sensor and thus also of the object can be determined. The established displacement can then be seen relative to the starting position. The current position can thus be determined together with the starting position. Acceleration sensors are already known in the art and may be used for the purposes of this disclosure.

According to one aspect, the carrier unit additionally comprises at least one magnetic field sensor. Defining the starting position comprises:
arranging the object region with the carrier unit outside the examination tunnel;
with the main magnet of the magnetic resonance tomography system switched on: measuring the magnetic field with the at least one magnetic field sensor;
drawing a conclusion as to the position of the object region on the basis of the measured magnetic field.

Arranging the object region with the carrier unit outside the examination tunnel is carried out, in particular, in such a way that the object region is arranged prior to entry into the examination tunnel, preferably on a patient table. The magnetic field sensor may be arranged on and/or fastened to the carrier unit at a firmly defined position and/or with a firmly defined orientation on the carrier unit. The magnetic field sensor may be a three-dimensional magnetic field sensor. A three-dimensional magnetic field sensor is a magnetic field sensor that is able to acquire a magnetic field strength for each of three spatial directions. The magnetic field sensor may, for example, be a Hall sensor. Three Hall elements may, for example, be provided, each of which can determine a magnetic field strength in a spatial direction. Alternatively, integrated 3D Hall sensors may be used. Three-dimensional Hall sensors, in particular, allow all three spatial directions of the magnetic field or of the magnetic flux density of different portions of a sensor circuit, in particular, sensor IC, to be acquired. A motion sensor may be assigned to the magnetic field sensor. The magnetic field sensor and the assigned motion sensor are preferably arranged together on a rigid plane of the carrier unit. The assigned motion sensor may, in particular, be an acceleration sensor. The assigned motion sensor may be used to track a change in the position of the magnetic field sensor or of the carrier unit, for example, even in the examination tunnel. The motion sensor may preferably be arranged next to, on, or below the magnetic field sensor. The magnetic field outside the examination tunnel is typically non-uniform and dependent on a distance from the examination tunnel. The position of the magnetic field sensor and thus the position of the carrier unit and thus the position of the object region may be associated with the measured magnetic field. In principle, the magnetic field is generally less, the greater the distance from the examination tunnel. With the assistance of information about the magnetic field distribution outside the magnetic field tunnel, a conclusion can thus be drawn as to the position of the object region in connection with the measured magnetic field. A z position of the object region can, for example, be determined with the assistance of the magnetic field. The z position can be determined from a magnitude of the measured magnetic field. The magnitude is, in particular, independent of the orientation of the sensor. For example, further sensors (e.g., acceleration sensors) may be used to establish the x and y coordinates. In particular, the acceleration sensors are used to determine an x coordinate and/or a y coordinate of the object region. The x coordinate and/or a y coordinate can be determined with the assistance of gravitation. It may, for example, be assumed that an x extent is horizontally oriented and a y extent is vertically oriented, i.e., parallel to the earth's gravitational field. Further position information can be determined with one or more further magnetic field sensors, for example, two further magnetic field sensors. The further magnetic field sensors may be arranged at a firmly defined position on the carrier unit. Advantageously, the firmly defined position may be used to define a relative positional relationship with regard to the positions of the first magnetic field sensor and the carrier unit. The further magnetic field sensors may be three-dimensional magnetic field sensors. The further magnetic field sensors may be Hall sensors, for example. A motion sensor may be assigned to one or more, preferably all, of the further magnetic field sensors. The further magnetic field sensors are preferably arranged in each case together with the assigned motion sensor in each case on a rigid plane of the carrier unit. The assigned motion sensors may be used to determine a change in position of the magnetic field sensors, in particular, also in the examination tunnel. The assigned motion sensors may be arranged next to, on, or below the respective magnetic field sensor. Provision may be made for a plurality of starting positions to be determined. To this end, provision may be made for the position to be determined for multiple positions of the patient couch and for the patient couch to be moved therebetween. Using multiple starting positions may enable a more precise determination of the position.

According to one aspect, defining the starting position further comprises: determining an orientation of the object region with at least one orientation sensor, in particular, by measuring a static acceleration caused by gravitational force. The orientation sensor may, in particular, be an acceleration sensor that is arranged and/or fastened on the carrier unit. An acceleration sensor may be used to determine the orientation of the earth's gravitational field. The orientation sensor may be one of the motion sensors or the at least one motion sensor. Advantageously, an acceleration sensor may, for example, be used both to determine an orientation of the starting position and to modify the position after the starting position has been determined. The orientation sensor may be assigned to a magnetic field sensor. The orientation sensor can thus determine the orientation of the magnetic field sensor. The magnetic field sensor and the assigned orientation sensor are preferably arranged together on a rigid plane of the carrier unit. Optionally, an external orientation sensor that is not arranged on the carrier unit may be provided. The orientation sensor may, for example, operate on the basis of optical acquisition. The orientation sensor may, for example, be a camera configured to acquire an orientation of the carrier unit and/or of the magnetic field sensor. Owing to the magnetic field outside the examination tunnel typically being rotationally symmetrical about an axis of the examination tunnel, it is not possible to use the magnetic field to define an orientation precisely. The orientation sensor can be of assistance in this respect, as it can establish not only a position but also an orientation of the magnetic field sensor and/or the carrier unit. The orientation of the object region can ultimately also be determined in this way.

According to one aspect, the carrier unit is arranged according to a defined geometric shape, in particular, cylindrically, around the object region. The carrier unit may be configured to assume the defined geometric shape automatically or to exhibit the defined geometric shape in an at least in part unmodifiable manner. The defined geometric shape may adapt itself, for example, flexibly to the object region or the anatomy. The defined geometric shape may correspond to the shape of the object region or be adapted to the shape of the object region. The cylindrical shape may be achieved, for example, by wrapping around the object region. The carrier unit may be of planar configuration and adaptable to the defined geometric shape. The carrier unit may, for example, be wound around a joint, in particular, around a knee or around an elbow. The carrier unit may, for example, be tubular and arranged around the object region by being drawn thereover. As a result of the defined geometric shape, movement determination and/or determination of the starting position may be particularly comprehensively and precisely possible. With a plurality of motion sensors in particular, complex movements may also be acquired in connection with the defined geometric shape. A cylindrical shape of the carrier unit may, for example, also replicate rotations of joints, such as a knee or an elbow.

According to one aspect, at least one central sensor, in particular, comprising a central magnetic field sensor and/or a central motion sensor, viewed in the direction of an object axis of the geometric shape, in particular, in the direction of a cylinder axis, is arranged centrally on the object region. The central motion sensor may, in particular, be a central acceleration sensor. The orientation of the object region may correspond to the orientation of an axis of the central acceleration sensor, wherein the axis extends, in particular, along the cylinder axis. The central motion sensor may be used, for example, to determine an orientation of a longitudinal axis of the object region, which may, in particular, correspond to the cylinder axis, or to track modification thereof. Additionally or alternatively, position determination of the object region may be determined and/or assisted by the central motion sensor. The central magnetic field sensor, for example, makes it possible, in particular, particularly precisely, to determine a starting position of the object region.

According to one aspect, the current position of the object region is determined by drawing a circle that runs through the center of the object region and corresponds to a cross-section of the geometric shape, in particular, of the cylinder, wherein the circle is drawn with the help of sensor data from the central sensor and with sensor data from two further sensors, in particular, from two further magnetic field sensors and/or motion sensors, projected onto the plane of the circle. The two further magnetic field sensors may be Hall sensors. The two further magnetic field sensors may, in particular, be three-dimensional magnetic field sensors. The two further motion sensors may, in particular, be acceleration sensors, in particular, two further motion sensors, and the central motion sensor may be acceleration sensors. The acceleration sensors may be used to determine an orientation of the magnetic field sensors. The motion sensors or acceleration sensors may be used to achieve movement tracking of the magnetic field sensors. To determine the circle, it may be assumed, for example, that the three sensors lie on an ellipse. Assuming that the central sensor already lies on the circle (since both the central sensor and the circle are central relative to the object region), the positions of the further sensors are projected onto the circle with known geometric considerations. The circle may thus advantageously be defined and thus also localized using the sensors. Using the circle, it is, in turn, possible to draw a conclusion as to the position and orientation of the object region.

According to one aspect, a position of the object region is determined by way of the center point of the circle. Since the circle lies centrally on the longitudinal axis of the object region and additionally may correspond at least approximately to the cross-section of the object region, it is possible, using the center point of the circle, also to determine the center point of the object region. While as a rule no sensor can be placed at the center point of the object region itself because this is not generally accessible for this purpose (for example, it is inside a knee), it is possible to draw a position of the center point of the object region by drawing a circle and with the assistance of the at least three sensors.

According to one aspect, a size of the object region is determined by way of a radius of the circle. It is thus advantageously possible also to establish the size of the object region by way of the automatically determinable circle. This may be advantageous, in particular, in the case of objects that can change size, but also for defining the size as a basis. Optionally, a starting size corresponding to the starting position may also be determined. In this case, it is also possible to track size in a similar way to movement.

According to one aspect, an orientation of the object region is determined with the assistance of an axis of the at least one central sensor, which axis runs in a defined orientation, in particular, parallel, to an object axis, in particular, cylinder axis. The object axis is, in particular, an object axis of the object region. If the object region is, for example, (approximately) cylindrical, the object axis may correspond to the axis of the cylinder. The object axis of the object region may correspond to an axis of the defined geometric shape of the carrier unit, in particular, the cylinder axis of a cylindrical shape of the carrier unit. The orientation of the axis of the central sensor relative to the geometric shape may be defined by fastening the motion sensor to the carrier unit. By suitably arranging the carrier unit around the object region, the axis of the carrier unit may be aligned with the object axis. The axis of the carrier unit may, for example, be a cylinder axis of a carrier unit arranged cylindrically around the object region. In this example, the geometric shape of the carrier unit is a cylinder. The axis of the motion sensor may thus be linked with the orientation of the object region. The at least one central sensor may, in particular, have two further axes that are perpendicular to the first axis. In the case of a cylindrical carrier unit, the two further axes of the central sensor may, in particular, lie in a plane that elliptically intersects the cylinder. In the case, in particular, of at least two further sensors being provided in addition to the at least one central sensor, the axes of the further sensors have a defined relationship with the axis of the central sensor. The further sensors may be further magnetic field sensors and/or motion sensors as described herein.

A further aspect of the disclosure is a measuring device comprising a carrier unit, in particular, magnetic resonance tomography body coil, and at least one motion sensor, in particular, acceleration sensor, preferably at least three motion sensors, wherein the at least one motion sensor is fastened to the carrier unit, wherein the carrier unit is substantially cylindrical or can be shaped into a cylindrical shape. The motion sensor may, in particular, be a three-dimensional motion sensor. The acceleration sensor may, in particular, be a three-dimensional acceleration sensor. All the advantages and features of the method are applicable mutatis mutandis to the measuring device and vice versa.

According to one aspect, the measuring device comprises one or more magnetic field sensors which are fastened to the carrier unit. The magnetic field sensors may preferably be three-dimensional magnetic field sensors. The magnetic field sensors may be Hall sensors. Three magnetic field sensors may be provided, for example. At least one magnetic field sensor, in particular, at least one central magnetic field sensor, is preferably arranged together with at least one orientation sensor, in particular, an acceleration sensor, on a rigid plane of the carrier unit. The rigid plane may be a printed circuit board, for example. The printed circuit board may be a printed circuit board of a body coil, especially if the carrier unit is a body coil.

According to one aspect, the cylindrical shape of the carrier unit or the cylindrical shape into which the carrier unit may be shaped has a cylinder axis, wherein at least one of the sensors, in particular, a magnetic field sensor and/or motion sensor, is arranged substantially centrally on the carrier unit when viewed in the direction of the cylinder axis. Position determination may advantageously be simplified with a central arrangement. Provision may, in particular, be made for the central sensor to be arranged centrally on the object region.

According to one aspect, the carrier unit is fixed rigidly with regard to deformation and/or is fixable in the cylindrical shape. Advantageously, deformation of the carrier unit and, indeed, distortion of the object region during a scan can thus be prevented, so simplifying movement tracking.

According to one aspect, the carrier unit has a marker that marks at least one defined position on the cylinder axis, in particular, the central position. The marker advantageously simplifies the exact positioning of the carrier unit at/on the object region. For example, the marker may be or comprise a cross. The marker may comprise a pattern that is configured to correspond to the object region and/or to correspond to an intended positioning around the object region. The marker may, for example, define a longitudinal extent that is oriented along a longitudinal extent of the object region. The longitudinal extent may be oriented along an elbow. The longitudinal extent may, for example, be defined by an elongate box and/or a straight line. The marker may include a scale. The scale may be configured to allow an extent of the object region to be read off using the scale.

According to one aspect, the measuring device has further sensor elements. The further sensor elements may be configured to acquire further information, for example. Examples of further information are further position information, temperature, etc. MR markers may be provided, for example. Additionally or alternatively, one or more barrier elements, for example, electromagnetic shielding, may be provided on the carrier unit.

Small transmit and/or receive coils (TX and/or RX coils) may optionally be arranged on the carrier unit. The coils may be arranged relative to at least one marker. The coils may be wrapped around a marker.

A further aspect of the disclosure is a magnetic resonance tomography system comprising a measuring device as described herein and an examination tunnel, wherein the magnetic resonance tomography system is configured to track movement of an object region in the examination tunnel with the at least one motion sensor, in particular, using a method as described herein. All the advantages and features of the method and measuring device are applicable mutatis mutandis to the magnetic resonance tomography system and vice versa.

All the aspects described herein may be combined with one another unless explicitly stated otherwise.

### BRIEF DESCRIPTION OF THE DRAWINGS

Aspects are described below with reference to the appended figures.

FIG. 1 shows a flow chart of a method for tracking the movement of an object region in an examination tunnel of a magnetic resonance tomography system according to one aspect of the disclosure,

FIG. 2 shows a flow chart of a method for tracking the movement of an object region in an examination tunnel of a magnetic resonance tomography system according to a further aspect of the disclosure,

FIG. 3 shows a measuring device with a carrier unit according to one aspect of the disclosure,

FIG. 4 shows the measuring device according to the aspect of FIG. 3 spread out and wound into a cylinder,

FIG. 5 shows a magnetic resonance tomography system with a measuring device according to one aspect of the disclosure,

FIG. 6 shows a magnetic resonance tomography system with a measuring device according to the aspect of FIG. 5 and

FIG. 7 illustrates one option for calculating the current position, including the orientation, of the object region from sensor data according to one aspect of the disclosure.

### DETAILED DESCRIPTION

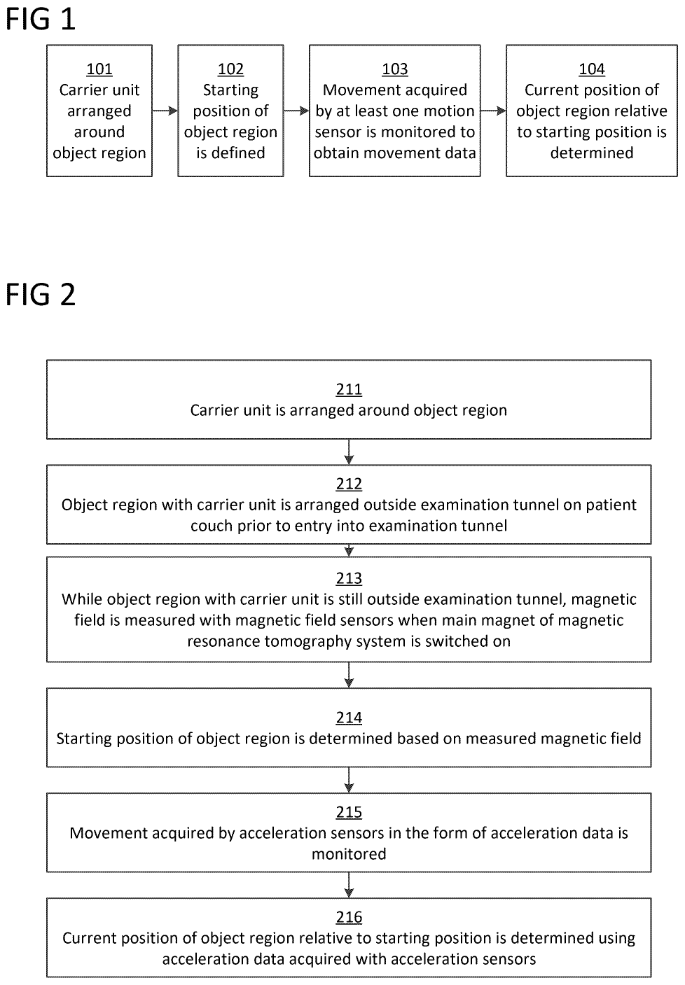

FIG. 1 shows a flow chart of a method for tracking the movement of an object region in an examination tunnel 6 of a magnetic resonance tomography system according to one aspect of the disclosure. In a first step 101, a carrier unit 1 is arranged around the object region such that the position of the carrier unit 1 is linked to the position of the object region. The carrier unit 1 may optionally be a magnetic resonance tomography body coil. In this case, the carrier unit 1 may advantageously be used both for the MRI measuring process and also for determining or tracking movement. At least one motion sensor is arranged on the carrier unit 1. A plurality of motion sensors, in particular, three motion sensors, may advantageously be arranged on the carrier unit 1. A plurality of motion sensors may enable more detailed movement tracking. The motion sensor or motion sensors may preferably be an acceleration sensor 3 or acceleration sensors 3. In a further step 102, a starting position of the object region is defined. The starting position may optionally include a starting orientation. The starting position may, for example, be defined by being input by a user, being received from an external device, and/or being established by a sensor system. In a further step 103, the movement acquired by the at least one motion sensor is monitored to obtain movement data. If the at least one motion sensor is an acceleration sensor 3, the movement data may be acceleration data of the acceleration sensor 3. In other words, acceleration of the object region may be detected and logged, preferably continuously. In a further step 104, a current position of the object region relative to the starting position is determined using the movement data acquired with the at least one motion sensor. The current position may preferably also encompass a current orientation. A conclusion can thus be drawn as to the absolute current position of the object region. If one or more acceleration sensors 3 are used, the current position can be determined by integrating the acceleration data twice over time.

FIG. 2 shows a flow chart of a method for tracking the movement of an object region in an examination tunnel 6 of a magnetic resonance tomography system according to a further aspect of the disclosure. In a first step 211, a carrier unit 1 is arranged around the object region such that the position of the carrier unit 1 is linked to the position of the object region. The carrier unit 1 may optionally be a magnetic resonance tomography body coil. Three acceleration sensors 3 and three magnetic field sensors 5 are arranged on the carrier unit 1. In each case one acceleration sensor 3 is assigned in each case to one magnetic field sensor 5, so resulting in three pairs, each of one acceleration sensor 3 and one magnetic field sensor 5. The pairs are, in each case, arranged on a rigid surface of the carrier unit 1. It may, in this way, be ensured that the orientation of the sensor pairs relative to one another is constant. In a further step 212, the object region with the carrier unit 1 is arranged outside the examination tunnel 6 on a patient couch 7 prior to entry into the examination tunnel 6. In a further step 213, while the object region with the carrier unit 1 is still outside the examination tunnel 6, the magnetic field is measured with the magnetic field sensors 5 when the main magnet of the magnetic resonance tomography system is switched on. Orientation in the earth's gravitational field is additionally measured with the acceleration sensors 3. In this case, the acceleration sensors 3 function as orientation sensors. In a further step 214, the starting position of the object region is determined on the basis of the measured magnetic field. With the magnetic field outside the examination tunnel 6 being dependent on the distance from the examination tunnel 6, it is possible to determine a z position, i.e., a distance from the examination tunnel 6 along the center axis of the examination tunnel 6. By using a plurality of magnetic field sensors 5, it is additionally possible to establish a distance from the center axis of the examination tunnel 6. Because the magnetic field is typically rotationally symmetrical outside the examination tunnel 6, it may be difficult or impossible also to determine an exact orientation solely using magnetic field measurement. The data from the acceleration sensors 3 is used for this purpose, said sensors allowing an orientation in the earth's gravitational field to be determined. It may also be possible to use the acceleration sensors 3 to assign an absolute orientation to the magnetic field sensors 5, whereby altogether, both the coordinate position and the orientation of the carrier unit 1 and, thus, of the object region may be determined. The established starting position thus comprises not only a coordinate position but also an orientation in space. In a further step 215, the movement acquired by the acceleration sensors 3 in the form of acceleration data is monitored, and the acceleration data is recorded or directly further evaluated in the next step. The acceleration data is acquired by the acceleration sensors 3. The acceleration sensors 3 may thus be used not just for establishing orientation of the starting position, but also for tracking movement, in particular, including in the examination tunnel 6. In this way, acceleration of the object region is preferably continuously detected and logged. In a further step 216, a current position of the object region relative to the starting position is determined using the acceleration data acquired with acceleration sensors 3. To this end, the acceleration data is integrated twice over time, so as to draw a conclusion as to any shift in location of the individual sensors. A conclusion can thus be drawn as to the absolute current position of the object region. The current position may preferably also encompass a current orientation. The acquired current positions of the sensors may be used to establish both a current coordinate position and a current orientation in space of the object region by taking account of the relative position of the sensors to one another.

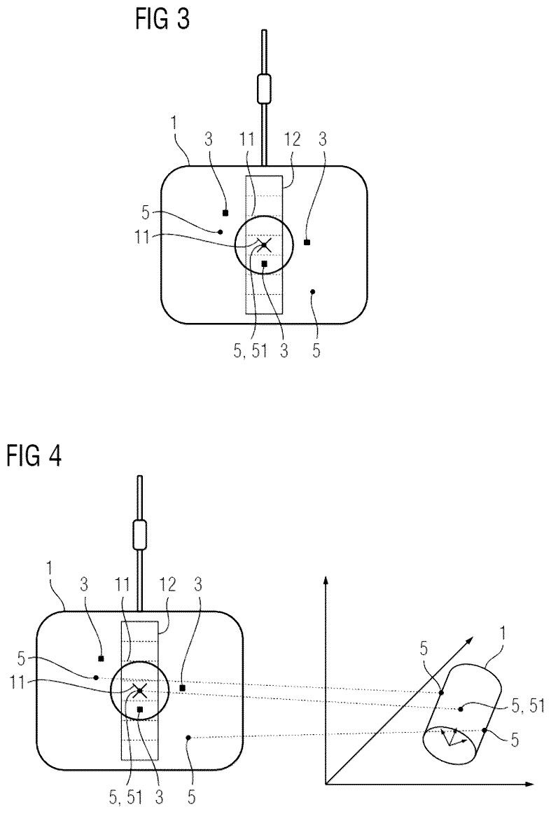

FIG. 3 shows a measuring device with a carrier unit 1 according to one aspect of the disclosure. The carrier unit 1 may, in particular, simultaneously serve as an MRI body coil. Three each of acceleration sensors 3 and magnetic field sensors 5 are arranged on the carrier unit 1. The carrier unit 1 has a marker that marks a central position on the cylinder axis. In this exemplary aspect, the marker comprises a cross 11. The cross 11 serves as orientation in order to ensure that the carrier unit 1 is arranged correctly or on the correct point around the object region. Provision is made, in particular, for the point with the cross 11 to be placed over a center of the object region. The cross 11 may, for example, be placed directly on a knee or elbow. The marker further comprises a scale 12, which may serve for orientation against an elongate object region, such as an elbow or knee region, and with which a length of the object region may additionally be measured off. The longitudinal extent of the scale 12 may, for example, be aligned with a longitudinal extent of an arm or a leg. The marker can thus assist with ensuring a correctly defined orientation and positioning of the carrier unit 1 relative to the object region.

FIG. 4 shows the measuring device with carrier unit 1 according to the aspect of FIG. 3, spread out and wound into a cylinder. In the cylindrical shape, the carrier unit 1 may, in particular, be arranged around an object region. The carrier unit 1 is configured such that it is rigidly fixable in the cylindrical shape, such that substantial deformation of the carrier unit 1 is prevented during an MRI scan. One of the magnetic field sensors 5 is a central magnetic field sensor 51, which is arranged substantially centrally on the cylinder axis of the carrier unit 1.

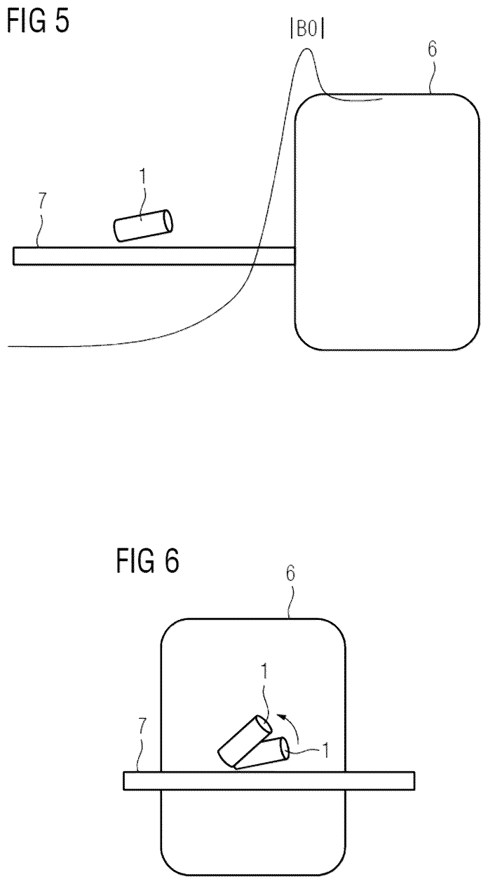

FIGS. 5 and 6 show a magnetic resonance tomography system with a measuring device according to one aspect of the disclosure. Said figures show the carrier unit 1 of the measuring device. The sensors of the measuring device are not shown in this depiction but may, for example, correspond to those shown in FIGS. 3 and 4. The magnetic resonance tomography system is configured to track the movement of an object region in an examination tunnel 6 of the magnetic resonance tomography system with the motion sensors, for example, acceleration sensors 3, which are fastened to the carrier unit 1. In FIG. 5, the carrier unit 1 is arranged outside the examination tunnel 6 around an object region which is not shown here. In particular, a patient is here lying on a patient couch 7, and the object region may be a part of the patient's body. This position may be understood as the starting position or home position of the patient couch 7. The carrier unit 1 is here already wrapped around the object region (not shown). In this position, when the main magnet of the magnetic resonance tomography system is switched on, the magnetic field sensors 5 can measure the magnetic field (B0) at the position of the magnetic field sensors 5. Using the magnitude of the magnetic field at the various magnetic field sensors 5, a conclusion can be drawn as to the positioning of the carrier unit 1 and of the object region in this starting position. Because, as a rule, the magnetic field is approximately rotationally symmetrical about an axis of the examination tunnel 6, at least one orientation sensor is used to determine the orientation of the starting position. The orientation sensor may be an acceleration sensor 3, with which the acceleration due to gravity may be measured, and thus, the orientation in the earth's magnetic field is determined.

If the patient couch 7 is then displaced with the object region and the carrier unit 1 into the examination tunnel 6, as shown in FIG. 6, measurement of the magnetic field is not very helpful for further position determination, because the magnetic field in the examination tunnel 6 is largely uniform. The acceleration sensors 3 or other motion sensors are then used to continue to track movement. By measuring the acceleration with acceleration sensors 3, it is possible through double integration to draw a conclusion as to changes in location. For this purpose, acceleration data is preferably captured without any interruption. If the change in location is thus added to the starting position, the respective current position can be determined. FIG. 6 shows a movement (along the arrow) and two corresponding different positions of the carrier unit 1. Using the collected movement data, it can, for example, be established whether an orientation of a field of view of the scan needs to be adjusted due to movement of the object region under examination.

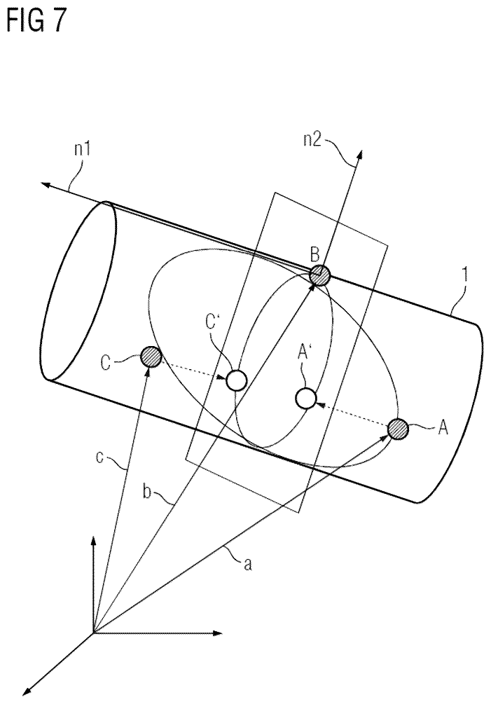

FIG. 7 illustrates one option for calculating the current position, including the orientation, of the object region according to one aspect of the disclosure. The position of the object region may be determined using the position of three sensors, which are arranged and fastened on the carrier unit 1. The three sensors may, for example, be magnetic field sensors 5. The magnetic field sensors 5 may each be linked with an acceleration sensor 3 in order, on the one hand, to determine more precisely the orientation of the magnetic field sensors 5 outside the examination tunnel 6 and, on the other hand, to be able also to track the movement of the magnetic field sensors 5 within the examination tunnel 6. The respective current position (a, b, c) of the sensors may be determined on the basis of the respective starting position (A, B, C) by simple addition of the recorded movement (D_A, D_B, D_C):
a=A+D_A
b=B+D_B
c=C+D_C

When using acceleration sensors 3, the movement (D_A, D_B, D_C) may, in each case, be determined by double integration on the basis of the acquired acceleration data. As soon as the current position of the three sensors is determined, the position can be determined, including the orientation of the cylindrical carrier unit 1, as a result of which a conclusion as to the position of the object region can, in turn, be drawn directly. In this respect, FIG. 7 shows the cylindrical carrier unit 1 with the sensors A, B, C, wherein the current position of the sensors is defined by the vectors a, b, c. The sensors A, B, C lie on a plane which intersects the cylinder in the form of an ellipse. The sensors A, B, C additionally lie on the surface of the cylinder. The central sensor B, which, viewed in the direction of the cylinder axis, is arranged centrally in the middle, is oriented such that a first axis n1 of the central sensor matches the orientation of the cylinder or is oriented parallel to the cylinder axis. A plane defined by the other two axes (n2, n3) of the central sensor intersects circularly with the cylinder, which corresponds to the circular cross-section of the cylinder at the position of the central sensor. To calculate the position and size of the circle, the positions (A, C) of the other two sensors along the axis n1 are projected onto the plane of the circle, so resulting in the points A′ and C′ lying on the circumference of the circle with the vectors a′ and c′ (using the straight line equations a+λ_A×n1 and c+λ_C×n1):

a′=intersection_of_the_plane_with_the_straight line(plane(n2,n3),straight line(a+λ_A×n1))

c′=intersection_of_the_plane_with_the_straight line(plane(n2,n3),straight line(c+λ_C×n1))

Using the coordinates of the points (a′, b, c′) on the circumference of the circle, it is, in turn, possible to determine the parameters of the circle, comprising the center of the circle and its radius. In this way, the position and size of the object region are determined. The position of the object region corresponds to the position of the center of the circle. The orientation of the object region is defined by n1. The size of the object region is determined in cross-section by the radius of the circle and may be measured in the longitudinal direction, for example, using a marker on the carrier unit 1 or in another manner or be retrieved, for example, from a database. Alternatively, the extent may also be established in the longitudinal direction, for example, using a statistical body model (for example, on the basis of the body size of the patient under examination), as is known in the prior art.

1. A method for tracking a movement of an object region in an examination tunnel of a magnetic resonance tomography system, wherein the method comprises:
a) arranging a carrier unit around the object region, such that a position of the carrier unit is linked to the position of the object region, wherein at least one motion sensor is arranged on the carrier unit;
(b) defining a starting position of the object region;
(c) monitoring the movement acquired by the at least one motion sensor in order to obtain movement data; and
(d) determining a current position of the object region relative to the starting position based on the movement data acquired with the at least one motion sensor.

2. The method as claimed in claim 1,
wherein the at least one motion sensor is at least one acceleration sensor and the movement data is acceleration data, and
wherein the current position of the object region is determined by integrating the acceleration data twice over time.

3. The method as claimed in claim 1,
wherein the carrier unit additionally comprises at least one magnetic field sensor, and
wherein the defining the starting position comprises:
arranging the object region with the carrier unit outside the examination tunnel;
when the main magnet of the magnetic resonance tomography system is switched on, measuring the magnetic field with the at least one magnetic field sensor; and
drawing a conclusion as to the position of the object region based on the measured magnetic field.

4. The method as claimed in claim 3, wherein the defining the starting position further comprises:
determining an orientation of the object region with at least one orientation sensor,
wherein the at least one motion sensor comprises the at least one orientation sensor.

5. The method as claimed in claim 1, wherein the carrier unit is arranged according to a defined geometric shape around the object region.

6. The method as claimed in claim 5, wherein at least one central sensor viewed in a direction of an object axis of the geometric shape is arranged centrally on the object region.

7. The method as claimed in claim 6,
wherein the current position of the object region is determined by drawing a circle that runs through the center of the object region and corresponds to a cross-section of the geometric shape, and
wherein the circle is drawn with help of sensor data from the at least one central sensor and with sensor data from at least two further sensors projected onto a plane of the circle.

8. The method as claimed in claim 7, wherein a position of the object region is determined by way of the center point of the circle.

9. The method as claimed in claim 7, wherein a size of the object region is determined by way of a radius of the circle.

10. The method as claimed in claim 7, wherein an orientation of the object region is determined with assistance of an axis of the at least one central sensor, which axis runs parallel to an object axis.

11. A measuring device, comprising:
a carrier unit and at least one motion sensor,
wherein the at least one motion sensor is fastened to the carrier unit, and the carrier unit is substantially cylindrical or can be shaped into a cylindrical shape.

12. The measuring device as claimed in claim 11, wherein the measuring device comprises one or more magnetic field sensors which are fastened to the carrier unit.

13. The measuring device as claimed in claim 11, wherein the cylindrical shape of the carrier unit or the cylindrical shape into which the carrier unit may be shaped has a cylinder axis, wherein at least one of the motion sensors is arranged substantially centrally on the carrier unit when viewed in a direction of the cylinder axis.

14. The measuring device as claimed in claim 11, wherein the carrier unit has a marker that marks at least one defined position on a cylinder axis.

15. A magnetic resonance tomography system comprising a measuring device comprising:
a carrier unit and at least one motion sensor, wherein the at least one motion sensor is fastened to the carrier unit, and the carrier unit is substantially cylindrical or can be shaped into a cylindrical shape; and
an examination tunnel, wherein the magnetic resonance tomography system is configured to track movement of an object region in the examination tunnel with the at least one motion sensor using the method as claimed in claim 1.
